(19) United States
(12) Patent Application Publication
(10) Pub. No.: US 2025/0143597 A1
(43) Pub. Date: May 8, 2025

(54) APPARATUS, METHOD AND SYSTEM FOR PROVIDING TACTILE MEDIUM

(71) Applicant: SEOUL NATIONAL UNIVERSITY R&DB FOUNDATION, Seoul (KR)

(72) Inventors: Hyoung KIM, Seoul (KR); Seong Hwan HWANG, Seoul (KR)

(21) Appl. No.: 18/282,298

(22) PCT Filed: May 26, 2023
(86) PCT No.: PCT/KR2023/007315

§ 371 Date: Dec. 13, 2024

(30) Foreign Application Priority Data
May 30, 2022 (KR) 10-2022-0066016
Aug. 4, 2022 (KR) 10-2022-0097419

(51) Int. Cl. A61B 5/055; A61B 5/00; G01R 33/48

(52) U.S. Cl. CPC A61B 5/055; A61B 5/0042; A61B 5/742; A61B 5/7455; G01R 33/4806

(57) ABSTRACT

An apparatus for providing a tactile medium according to an embodiment includes a tactile medium unit configured to receive a plurality of tactile media capable of stimulating tactile sensation of a subject, a medium driving unit configured to move the plurality of tactile media so as to provide a specific tactile medium among the plurality of tactile media, and a drive controller configured to drive the medium driving unit so as to provide the specific tactile medium to the subject.

### BACKGROUND

### 1. Technical Field

The present invention relates to an apparatus, method and system for providing a tactile medium, and more particularly to an apparatus, method and system for providing a tactile medium, which are applicable to magnetic resonance imaging equipment.

### 2. Background Art

Researches for identifying perception and cognitive characteristics of the brain according to tactile sensation using functional magnetic resonance imaging (fMRI) techniques In order to conduct research on human have been conducted. tactile in an environment equipped with magnetic resonance imaging equipment, an apparatus for providing the tactile sensation should be operated stably without being affected by the magnetic resonance imaging equipment, as well as, the apparatus should not affect the magnetic resonance imaging equipment.

Research on the fMRI related to the tactile sensation is mainly intended to stimulate the tactile sensation with vibration, thus to analyze the size, region and pattern, etc.

of brain activation. In order to apply the tactile stimulation with vibration in the environment equipped with the magnetic resonance imaging equipment, a piezoelectric element is mainly used.

Meanwhile, there is an apparatus for providing specific Braille to the fMRI image as a tactile medium to measure the brain activity of blind people, but the conventional apparatus for providing Braille is intended to provide only specific Braille as a stimulation medium, such that its versatility for brain activity researches according to various tactile media is very low, and it is not possible to measure an exact point in time when a finger moves to touch the Braille, as well as there are problems entailed in high price and durability using a piezoelectric actuator.

### SUMMARY

An object of the present invention is to provide an apparatus, method and system for providing a tactile medium, which are capable of providing various tactile media, as well as applicable to magnetic resonance imaging equipment.

To achieve the above object, according to an aspect of the present disclosure, there is provided an apparatus for providing a tactile medium including: a tactile medium unit configured to receive a plurality of tactile media capable of stimulating tactile sensation of a subject; a medium driving unit configured to move the plurality of tactile media so as to provide a specific tactile medium among the plurality of tactile media; and a drive controller configured to drive the medium driving unit so as to provide the specific tactile medium to the subject.

The medium driving unit may move the plurality of tactile media using an air actuator.

The medium driving unit may further include a rail, wherein the plurality of tactile media may be formed in a cubic block and fixed to the rail, respectively, and the air actuator may convert a pressure energy of compressed air into a mechanical energy to cause the rail to be linearly moved or rotationally moved, thereby moving the tactile media.

The medium driving unit may further include an air compressor configured to compress air and air valves configured to control air flow from the air compressor to the air actuator, wherein the drive controller may be configured to control a sequence in which the air valves are sequentially opened to move teeth of pistons of the air actuator back and forth.

The apparatus for providing a tactile medium may further include a medium providing unit which includes a medium providing case having a finger insert hole for providing a specific tactile medium moved by the medium driving unit to the subject.

The medium providing unit may further include a medium contact detection sensor configured to detect a point in time when a finger touches the specific tactile medium moved by the medium driving unit.

The medium contact detection sensor may include a laser transmission element and a laser reception element.

In addition, according to another aspect of the present disclosure, there is provided a system for providing a tactile medium including: the apparatus for providing a tactile medium according to the aspect having the above-described configurations; and a position adjustment table on which the apparatus for providing a tactile medium is placed, and having adjustable height and angle.

The system for providing a tactile medium may further include: a visual presentation device configured to present a visual pattern on a screen; and magnetic resonance imaging equipment configured to take images of a brain using a magnetic field generated by a magnetic force, wherein, when contact with a specific tactile medium is detected by the apparatus for providing a tactile medium according to a visual pattern presented by the visual presentation device, the magnetic resonance imaging equipment records a point in time of contact with the specific tactile medium in the brain image scanned by the magnetic resonance imaging equipment.

Further, according to another aspect of the present disclosure, there is provided a method for providing a tactile medium including: receiving a plurality of tactile media capable of stimulating tactile sensation of a subject; moving the plurality of tactile media so as to provide a specific tactile medium among the plurality of tactile media; and providing the specific tactile medium to the subject.

The moving step may include moving the plurality of tactile media using an air actuator.

The method for providing a tactile medium may further include detecting whether a finger of the subject comes into contact with the specific tactile medium.

The method for providing a tactile medium may further include, when it is detected that the finger has come into contact with the specific tactile medium in the detecting step, recording a point in time of contact with the specific tactile medium in the brain image scanned by the magnetic resonance imaging equipment.

According to the present invention having the above-described configurations, it is possible to electively and easily present tactile media capable of feeling various tactile sensations one by one.

In addition, according to the present invention, it is possible to detect a finger contacting with the tactile medium using an optical sensor.

Further, according to the present invention, it is also possible to manufacture an air actuator, and the like by using a 3D printer, thereby securing a reasonable price and ease of maintenance.

Furthermore, the present invention allows researchers to conduct basic neurophysiology research on tactile sensation targeted at the normal people and research on tactile neurological disease targeted at the patients, for example, research on tactile sensation, pain-related brain activity and cognitive behavior in the general public and patients through fMRI images.

### BRIEF DESCRIPTION OF DRAWINGS

FIG. 1 is a block diagram schematically illustrating an apparatus for providing a tactile medium according to an embodiment of the present disclosure.

FIG. 2 is a block diagram specifically illustrating an apparatus for providing a tactile medium according to another embodiment of the present disclosure.

FIG. 3A is a perspective view exemplarily illustrating an air actuator and a rail of the medium driving unit shown in FIG. 2, and FIG. 3B is a front view exemplarily illustrating a combination of the tactile medium unit and the rail shown in FIG. 2.

FIG. 4 is a perspective view exemplarily illustrating the medium providing unit shown in FIG. 2.

FIG. 5 is a block diagram specifically illustrating a system for providing a tactile medium according to another embodiment of the present disclosure.

FIG. 6 is a perspective view exemplarily illustrating a position adjustment table shown in FIG. 5.

FIG. 7 is a flowchart illustrating a method for providing a tactile medium according to another embodiment of the present disclosure.

### DETAILED DESCRIPTION

Hereinafter, embodiments will be described in detail with reference to the accompanying drawings. In denoting reference numerals to components of respective drawings, it should be noted that the same components will be denoted by the same reference numerals although they are illustrated in different drawings. Further, in description of preferred embodiments of the present invention, the publicly known functions and configurations related to the present invention, which are verified to be able to make the purport of the present invention unnecessarily obscure will not be described in detail.

Meanwhile, in respective steps, each of the steps may occur differently from the specified order unless a specific order is clearly described in the context. That is, each of the steps may be performed in the same order as the specified order, may be performed substantially simultaneously, or may be performed in the reverse order.

Embodiments of the present disclosure are illustrated for the purpose of describing the technical idea of the present disclosure. The scope of rights according to the present disclosure is not limited to the embodiments provided below or specific descriptions of these embodiments.

All technical terms and scientific terms used in the present disclosure have meanings commonly understood by a person who has a common knowledge in the technical field to which the present disclosure belongs, unless otherwise defined. All terms used in the present disclosure are selected for the purpose of more clearly describing the present disclosure and are not selected to limit the scope of rights according to the disclosure.

The expressions such as “comprising,” “including,” “having,” and the like used in the present disclosure should be understood as open-ended terms that imply the possibility of including other embodiments, unless otherwise stated in the phrase or sentence including the corresponding expression.

Expressions the singular forms described in the present disclosure may include plural meanings unless the context clearly indicates otherwise, and this is applied equally to the expressions in the singular forms described in the claims.

Hereinafter, embodiments of the present disclosure will be described with reference to the accompanying drawings.

Further, in the description of the following embodiments, the same or corresponding components may not be described in overlapping manner. However, although the corresponding components are not described, it does not intend that these components are excluded in any embodiment.

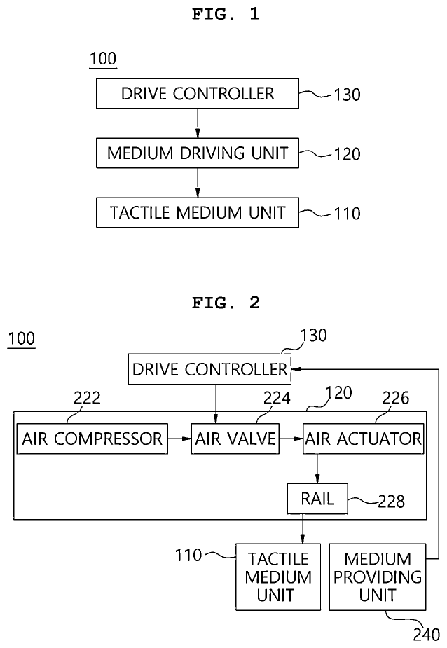

FIG. 1 is a block diagram schematically illustrating an apparatus for providing a tactile medium according to an embodiment of the present disclosure.

As shown in FIG. 1, an apparatus for providing a tactile medium (“tactile medium providing apparatus”) 100 includes a tactile medium unit 110, a medium driving unit 120 and a drive controller 130.

The tactile medium unit 110 includes a plurality of tactile media capable of stimulating tactile sensation of a subject. The plurality of tactile media may be, for example, natural media such as hair, stone and leather, etc., and may be artificial media artificially made by using a 3D printer or the like. In addition, the plurality of tactile media may be artificial media of Braille letters for providing tactile stimulation.

The medium driving unit 120 will physically move the plurality of tactile media to provide one specified tactile medium. To this end, the medium driving unit 120 may physically move the plurality of tactile media using an air actuator.

The drive controller 130 controls and drives the medium driving unit 120 so as to provide one tactile medium to the subject.

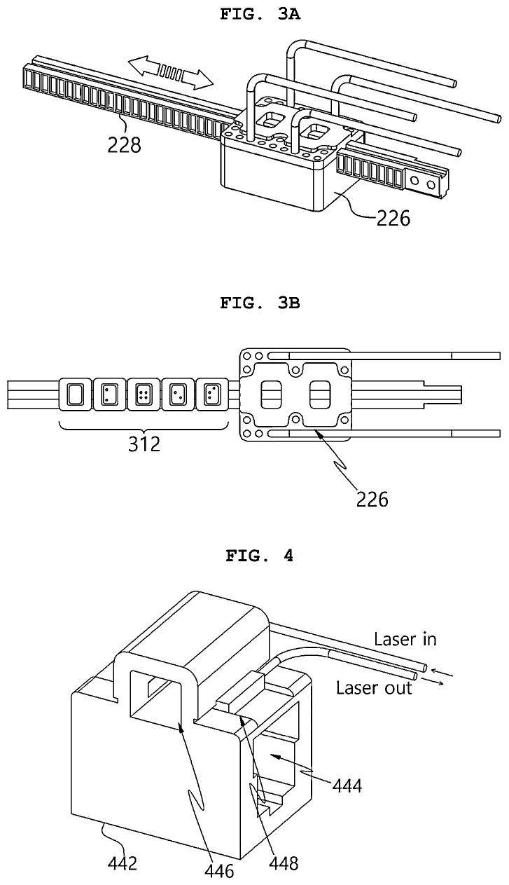

FIG. 2 is a block diagram specifically illustrating an apparatus for providing a tactile medium according to another embodiment of the present disclosure, FIG. 3A is a perspective view exemplarily illustrating an air actuator and a rail of the medium driving unit shown in FIG. 2, FIG. 3B is a front view exemplarily illustrating a combination of the tactile medium unit and the rail shown in FIG. 2, and FIG. 4 is a perspective view exemplarily illustrating the medium providing unit shown in FIG. 2.

As shown in FIG. 2, the tactile medium providing apparatus 100 includes a tactile medium unit 110, a medium driving unit 120, a medium providing unit 240 and a drive controller 130.

The tactile medium unit 110 is provided with a plurality of tactile media capable of stimulating tactile sensation of the subject. The plurality of tactile media may be formed in a cubic block to provide various types of tactile media, respectively. The cube blocks may be made of non-magnetic materials such as polyoxymethylene for compatibility with magnetic resonance imaging equipment.

Braille may be differently arranged on each of the plurality of tactile media. The Braille is widely used when the blind people or partially sighted people read letters by tactile sensation. In the present disclosure, 3×2 Braille cells are used to identify selective brain regions for tactile sensation. However, the plurality of tactile media may be, for example, natural media such as hair, stone and leather, etc., and may be artificial media artificially made by using a 3D printer or the like. Thereby, it is possible to confirm how the brain is activated through the tactile sensation felt by the subjects in nature.

The medium driving unit 120 will physically move the plurality of tactile media to provide one tactile medium. To this end, the medium driving unit 120 may include an air compressor 222, air valves 224, an air actuator 226 and a rail 228. FIG. 3A shows a perspective view exemplarily illustrating the air actuator 226 and the rail 228 of the medium driving unit shown in FIG. 2.

The air compressor 222 compresses air to produce and store compressed air at high pressure, and supplies the compressed air as needed. The compressed air produced by the air compressor 222 is supplied to the air actuator 226 through the air valve 224. The air actuator 226 converts a pressure energy of the compressed air into a mechanical energy to cause the rail 228 to be linearly moved. The rail 228 may be accurately moved to a specific position according to an operation of the air actuator 226. These air actuator 226 and rail 228, etc. may be manufactured by the 3D printer. FIG. 2 shows the rail 228 as an embodiment, but the plurality of tactile media may be provided by rotational motion of an actuator using a rotation plate.

Meanwhile, a plurality of tactile medium blocks 312 of the tactile medium unit 110 may be inserted into an upper notch of the rail 228 and fixed thereto with plastic screws through lower portions thereof. Accordingly, one of the plurality of tactile media may be moved along with the rail 228 and provided to the subject. FIG. 3B shows a front view exemplarily illustrating the combination of the tactile medium unit 110 and the rail 228 shown in FIG. 2.

The medium providing unit 240 may test a finger movement while providing a specific tactile medium to the subject. This medium providing unit 240 may include a medium providing case 442, a rail tunnel 444, a finger insert hole 446, and a medium contact detection sensor 448. The tactile medium blocks 312 on the rail 228 pass through the rail tunnel 444, and a tactile medium selected among them may be provided inside the finger insert hole 446. Thereby, it is possible to provide a specific tactile medium to the subject.

The medium contact detection sensor 448 may include a laser transmission element and a laser reception element in order to detect an exact point in time when the finger touches the tactile medium. The medium contact detection sensor 448 attached to the medium providing case 442 may detect an exact time when the finger touches the tactile medium block. The laser reception elements of the medium contact detection sensor 448 disposed on both sides of the finger insert hole 446 send a 5 V signal to the drive controller 130 since the laser beam is blocked when the finger is inserted into the finger insert hole 446 and comes into contact with the tactile medium block 312, and send a 0 V signal to the drive controller 130 when the finger comes out of the finger insert hole 446. Accordingly, the drive controller 130 may measure the time and point in time when the finger of the subject comes into contact with the tactile medium block 312. FIG. 4 shows the laser transmission element and the laser reception element as the medium contact detection sensor 448 as an example, but the medium contact detection sensor includes not only the laser element but also other optical elements using light, and may also include a contact sensor whose capacitance values are changed by contact.

The drive controller 130 controls and drives the medium driving unit 120 so as to provide one tactile medium to the subject through the medium providing unit 240. When any tactile medium is selected to be provided through an input interface (not shown) etc., the drive controller 130 may transmit information about which tactile medium has been provided to the air valve 224 to control the air actuator 226. The drive controller 130 may count the number of times the air valves 224 are sequentially opened to move teeth of pistons (not shown) of the air actuator 226 back and forth. The air valves 224 which are sequentially opened and closed according to the calculated results, i.e., the counted number of the drive controller 130 may control air flow from the air compressor 222 to the air actuator 226. The air flow controlled by the air valve 224 may move the teeth of two pistons of the air actuator 226. The sequential motion of the pistons of the air actuator 226 may be converted into a back and forth motion of the rail 228. Since the tactile medium blocks 312 are attached to the rail 228, the tactile medium blocks 312 may move in a front-rear direction along the rail 228.

The drive controller 130 may store the exact time when the subject moves his or her finger by providing the specific tactile medium block 312 as information on each event in a computer.

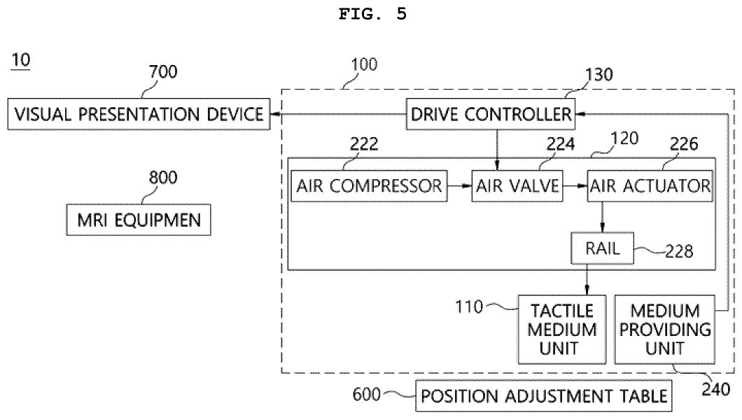

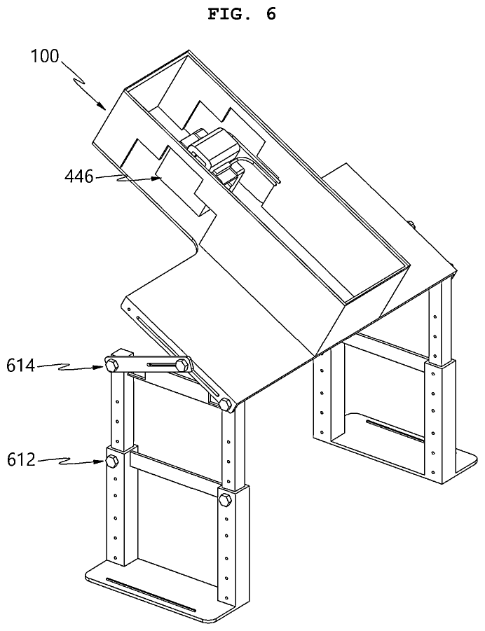

FIG. 5 is a block diagram specifically illustrating a system for providing a tactile medium according to another embodiment of the present disclosure, and FIG. 6 is a perspective view exemplarily illustrating a position adjustment table shown in FIG. 5

As shown in FIG. 5, a system for providing a tactile medium (“tactile medium providing system”) 10 includes a tactile medium providing apparatus 100, a position adjustment table 600, a visual presentation device 700, and magnetic resonance imaging (MRI) equipment 800.

The tactile medium providing apparatus 100 may include the tactile medium unit 110, a medium driving unit 120, a medium providing unit 240 and a drive controller 130. In the tactile medium providing apparatus 100 shown in FIG. 5, the components having the same configuration as those of the tactile medium providing apparatus 100 shown in FIG. 2 will not be described.

The tactile medium providing apparatus 100 is placed on the position adjustment table 600, and has adjustable height and position. That is, the height and angle of the position adjustment table 600 may be adjusted so that subjects may move their fingers comfortably and naturally.

The subject is required to minimize movement in order to examine brain activity while lying on a scanner table in the magnetic resonance imaging equipment 800. Due to the limitation in the movement, it is difficult for the subject to find a comfortable height and angle of the upper limb, which may vary depending on the individual's body shape. Therefore, the position of the tactile medium providing apparatus 100 should be adjusted so as to be placed in the most comfortable position for touching the tactile medium.

FIG. 6 shows a perspective view exemplarily illustrating the position adjustment table shown in FIG. 5. The tactile medium providing apparatus 100 may be mounted in the center of the position adjustment table 600. The position adjustment table 600 may include a height adjustment unit 612 for adjusting the height and an angle adjustment unit 614 for adjusting the angle. The subject may naturally come into contact with the tactile medium with his or her finger using this position adjustment table 600.

Meanwhile, the position adjustment table 600 may be positioned on the subject and fixed to a patient table (not shown) with plastic bolts.

The visual presentation device 700 presents a visual pattern on a screen. The visual presentation device 700 may use a projector and the screen in order to provide visual stimulation of when to bring into contact with the tactile medium during actual measurement.

The magnetic resonance imaging equipment 800 takes images of the brain using a magnetic field generated by a magnetic force.

The subject comes into contact with the specific tactile medium block 312 of the tactile medium providing apparatus 100 with his or her finger while lying on the scanner table of the magnetic resonance imaging equipment 800. When the medium contact detection sensor 448 of the medium providing unit 240 detects that the finger comes into contact with the tactile medium of the specific tactile medium block 312, the magnetic resonance imaging equipment 800 records the contact time of the specific tactile medium block 312 in the scanned brain image of the subject. Accordingly, it is possible to analyze a change in the brain activity due to the contact of the tactile medium block 312. That is, the brain image at the point in time of contact with the tactile medium may be analyzed by recording exact time information on the contact with the tactile medium at which point in time while acquiring brain images by scanning the brain.

The brain image in the magnetic resonance imaging equipment 800 may be acquired using only the tactile medium providing apparatus 100, but may also be acquired using the tactile medium providing apparatus 100 together with the visual providing device 700.

As described above, the tactile medium providing apparatus 100 may provide a specific tactile medium among various tactile media to the subject, and as a finger comes into contact with the specific tactile medium, a selectively activated brain region may be checked from the images scanned by the magnetic resonance imaging equipment 800.

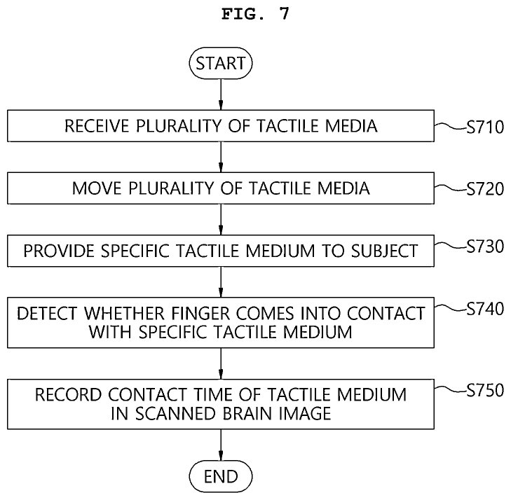

FIG. 7 is a flowchart illustrating a method for providing a tactile medium according to another embodiment of the present disclosure.

As shown in FIG. 7, the tactile medium unit 110 receives a plurality of tactile media capable of stimulating tactile sensation of the subject (S710). The plurality of tactile media may be formed in a cubic block to provide various types of tactile media, respectively. The cube blocks may be made of non-magnetic materials such as polyoxymethylene for compatibility with magnetic resonance imaging equipment 800.

The medium driving unit 120 moves the plurality of tactile media so as to provide a specific tactile medium among the plurality of tactile media (S720). To this end, the medium driving unit 120 may include the air compressor 222, the air valve 224, the air actuator 226 and the rail 228.

First, when any tactile medium is selected to be provided through an input interface (not shown) etc., the drive controller 130 may calculate a sequence in which the air valves 224 are sequentially opened to move the teeth of pistons (not shown) of the air actuator 226 back and forth. Then, the drive controller may transmit sequence information related to the selected tactile medium to the air valve 224 in order to control the air actuator 226. The air flow controlled by the air valve 224 may move the teeth of two pistons of the air actuator 226. The sequential motion of the pistons of the air actuator 226 may be converted into a back and forth motion of the rail 228. Since the tactile medium blocks 312 are attached to the rail 228, the tactile medium blocks 312 may move in a front-rear direction along the rail 228.

The medium providing unit 240 provides the specific tactile medium moved by the medium driving unit 120 to the subject (S730). The medium providing unit 240 may include the medium providing case 442, the rail tunnel 444 and the finger insert hole 446 in order to provide the specific tactile medium to the subject.

The medium providing unit 240 detects whether a finger comes into contact with the specific tactile medium while providing the specific tactile medium to the subject (S740). The medium contact detection sensor 448 attached to the medium providing case 442 may detect an exact point in time when the finger touches the tactile medium block.

When the medium contact detection sensor 448 of the medium providing unit 240 detects that the finger comes into contact with the tactile medium of the specific tactile medium block 312, the magnetic resonance imaging equipment records the contact time of the specific tactile medium block 312 in the scanned brain image of the subject (S750). Accordingly, it is possible to analyze a change in the brain activity due to the contact of the tactile medium block 312. The brain image in the magnetic resonance imaging equipment 800 may be acquired using only the tactile medium providing apparatus 100, but may also be acquired using the tactile medium providing apparatus 100 together with the visual providing device 700.

As described above, the tactile medium providing apparatus 100 may provide a specific tactile medium among various tactile media to the subject, and as a finger comes into contact with the specific tactile medium, a selectively activated brain region may be checked from the images scanned by the magnetic resonance imaging equipment 800.

Meanwhile, the present inventors have verified usability of the tactile medium providing apparatus 100 according to the embodiment of the present disclosure. The verification includes brain activity analysis by tactile sensation, brain activity analysis by visual sensation, and brain activity analysis by visual and tactile sensations.

In the brain activity analysis by the tactile sensation, the subjects were instructed to insert their fingers into the finger insert hole 446 of the medium providing unit 240 in order to bring them into contact with each Braille pattern without other visual stimuli using the visual stimulation. In the brain activity analysis by the visual sensation, the subjects were instructed to touch a flat surface with their fingers while looking at each fractal image displayed on the center of the screen. In the analysis of brain activity by the visual and tactile sensations, when the Braille pattern is changed from a blue pattern to a red pattern, the subjects were instructed to insert their fingers into the finger insert hole 446 of the medium providing unit 240 to bring them into contact with the Braille medium, and when the red pattern is changed back to the blue pattern, the subjects were instructed to remove the finger from the finger insert hole 446 of the medium providing unit 240.

According to the above-described verification, the subjects explicitly recognized the tactile and/or visual stimuli, and in fact, the subjects successfully recognized individual tactile media and exhibited that the level of tactile perception was similar to the level of visual perception. Further, when touching the tactile stimulation or seeing the visual stimulation using the magnetic resonance imaging equipment 800, the tactile processing region and the visual processing region of the brain were selectively activated, respectively.

The present invention has been described with reference to the preferred embodiments above, and it will be understood by those skilled in the art that various modifications may be made within the scope without departing from essential characteristics of the present invention. Accordingly, it should be interpreted that the scope of the present invention is not limited to the above-described embodiments, and other various embodiments within the scope equivalent to those described in the claims are included within the present invention.

1. An apparatus for providing a tactile medium, the apparatus comprising:
a tactile medium unit configured to receive a plurality of tactile media capable of stimulating tactile sensation of a subject;
a medium driving unit configured to move the plurality of tactile media so as to provide a specific tactile medium among the plurality of tactile media; and
a drive controller configured to drive the medium driving unit so as to provide the specific tactile medium to the subject.

2. The apparatus of claim 1, wherein the medium driving unit moves the plurality of tactile media using an air actuator.

3. The apparatus of claim 2, wherein the medium driving unit further comprises a rail,
wherein the plurality of tactile media are formed in a cubic block and fixed to the rail, respectively, and
the air actuator converts a pressure energy of compressed air into a mechanical energy to cause the rail to be linearly moved or rotationally moved, thereby moving the tactile media.

4. The apparatus of claim 3, the medium driving unit further comprises an air compressor configured to compress air and air valves configured to control air flow from the air compressor to the air actuator,
wherein the drive controller is configured to control a sequence in which the air valves are sequentially opened to move teeth of pistons of the air actuator back and forth.

5. The apparatus of claim 1, further comprising a medium providing unit which comprises a medium providing case having a finger insert hole for providing a specific tactile medium moved by the medium driving unit to the subject.

6. The apparatus of claim 5, wherein the medium providing unit further comprises a medium contact detection sensor configured to detect a point in time when a finger touches the specific tactile medium moved by the medium driving unit.

7. The apparatus of claim 6, wherein the medium contact detection sensor comprises a laser transmission element and a laser reception element.

8. A system for providing a tactile medium, the system comprising:
the apparatus of claim 6; and
a position adjustment table on which the apparatus for providing a tactile medium is placed, and having adjustable height and angle.

9. The system of claim 8, further comprising:
a visual presentation device configured to present a visual pattern on a screen; and
magnetic resonance imaging equipment configured to take images of a brain using a magnetic field generated by a magnetic force, wherein, when contact with a specific tactile medium is detected by the apparatus of a visual pattern presented by the visual presentation device, the magnetic resonance imaging equipment records a point in time of contact with the specific tactile medium in the brain image scanned by the magnetic resonance imaging equipment.

10. A method for providing a tactile medium comprising:
receiving a plurality of tactile media capable of stimulating tactile sensation of a subject;
moving the plurality of tactile media so as to provide a specific tactile medium among the plurality of tactile media; and
providing the specific tactile medium to the subject.

11. The method of claim 10, wherein the moving comprises moving the plurality of tactile media using an air actuator.

12. The method for of claim 11, wherein the moving further comprises moving the plurality of tactile media using a rail;
wherein the plurality of tactile media are formed in a cubic block and fixed to the rail, respectively, and
the air actuator converts a pressure energy of compressed air into a mechanical energy to cause the rail to be linearly moved or rotationally moved, thereby moving the tactile media.

13. The method of claim 12, wherein, in the moving, the plurality of tactile media are moved by further using an air compressor configured to compress air and air valves configured to control air flow from the air compressor to the air actuator, and
in the moving, a sequence in which the air valves are sequentially opened is controlled to move teeth of pistons of the air actuator back and forth.

14. The method of claim 10, further comprising detecting whether a finger of the subject comes into contact with the specific tactile medium.

15. The method of claim 14, further comprising, when it is detected that the finger has come into contact with the specific tactile medium in the detecting, recording a point in time of contact with the specific tactile medium in the brain image scanned by the magnetic resonance imaging equipment.

16. The method of claim 14, further comprising:
presenting a visual pattern on a screen; and
when it is detected that the finger has come into contact with the specific tactile medium in the detecting according to the visual pattern presented in the presenting, recording a point in time of contact with the specific tactile medium in the scanned brain image using magnetic resonance imaging equipment.
